(19) United States
(12) Patent Application Publication
(10) Pub. No.: US 2003/0073124 A1
(43) Pub. Date: Apr. 17, 2003

(54) System, method, and apparatus for submitting genetic samples and receiving genetic testing results anonymously

(75) Inventors: Brad Bowman, Portland, OR (US); Philip Marshall, Portland, OR (US)

Correspondence Address: Kolisch Hartwell, P.C., 200 Pacific Building, 520 S.W. Yamhill Street, Portland, OR, 97204, US

(73) Assignee: Genacy

(21) Appl. No.: 10/268,211

(22) Filed: Oct. 9, 2002

Related U.S. Application Data
(60) Provisional application No. 60/328,864, filed on Oct. 11, 2001.

(51) Int. Cl. C12Q 1/68; G06F 17/60; G06F 19/00; G01N 33/48; G01N 33/50

(52) U.S. Cl. 435/6; 702/20; 705/2

(57) ABSTRACT

Systems, apparatus, and methods for anonymously testing and reporting drug efficacy or safety are provided. One method described herein includes providing a DNA collection kit configured to enable collection and forwarding of a DNA test specimen for testing, wherein the kit includes a user results request with a user results identifier. The method further includes receiving the user results request, receiving test results for the DNA test specimen, wherein the DNA test specimen is identified by a computer-readable specimen identifier associated with the user results identifier, matching the specimen identifier for the test results of the DNA test specimen with the associated user results request via the user results identifier, and forwarding the test results in accordance with the user results request.

### CROSS-REFERENCE TO RELATED APPLICATIONS

This application claims priority from U.S. Provisional Patent Application Serial No. 60/328,864 of Brad Bowman and Philip Marshall, for A SYSTEM AND METHOD FOR SUBMITTING GENETIC SAMPLES AND RECEIVING GENETIC TESTING RESULTS ANONYMOUSLY, filed Oct. 11, 2001, the disclosure of which is hereby incorporated by reference.

### TECHNICAL FIELD

The present invention relates generally to the field of genetic testing. More particularly, the present invention provides a method and system for submitting genetic test samples and receiving the respective test results anonymously.

### BACKGROUND OF THE INVENTION

The promise of personalized medicine through the use of personal genetic information to predict responsiveness to drug therapy is matched only by the fear that personal privacy will be lost if this and other genetic information is improperly disclosed. Patients are becoming more self-directed, more aware of advances in technology, more aware of variations in drug effectiveness and safety, and more concerned about personal privacy. Educated consumers who desire more effective and safe drugs will direct the patient-centric healthcare system of the 21^{st} century. The most convenient way to achieve this goal will be to check the patient's genetic make-up for markers associated with drug safety and effectiveness. However, the notion of genetic testing conjures up concerns over personal privacy. These concerns may be justified given the discrimination and stigmatization that could occur if identifiable personal genetic information is disclosed.

In an effort to support more “personalized” drug therapies and to reduce liability, pharmaceutical companies will increasingly focus on identifying individuals who are likely to be the most appropriate and responsive to their respective drugs.

Biotechnology and pharmaceutical companies, academic institutions and private research facilities are discovering new genetic markers daily, helping to identify traits associated with variations in drug effectiveness and safety. One application for this technology may be to increase the approval for new compounds. The United States Food and Drug Administration (FDA) currently rejects approximately 80% of all new drug applications (NDAs) because of safety or efficacy concerns. The result of this is that most compounds that would be safe and effective for many individuals are not currently being made available to those individuals. It is hypothesized that more compounds would be deemed approvable if a reliable system and method existed to readily identify sub-populations of patients for whom such compounds would be safe and effective.

Another intended application of this technology is to enhance patient safety. Presently physicians prescribe drugs largely by trial and error. This is because most drugs are manufactured “off the rack” to be safe and effective for as many people as possible, rather than targeted to individuals. This imprecision in prescribing drugs has resulted in an enormous capacity for adverse drug reactions. Adverse drug reactions (ADRs) to prescription drugs have been reported to be responsible for the deaths of approximately 106,000 Americans each year—roughly three times as many as are killed by automobiles. Extending these genetic marker tests directly to the point of care can help identify patients for whom existing “off the rack” or future “personalized” medications will be both safe and effective. Because of the enormous benefit to society, it is likely that regulatory agencies will require such provisions in the near future.

Genetic testing is emerging as one way in which patients will be screened for appropriate drug therapy in the future, but special privacy concerns must first be addressed. Today, genetic testing, prior to the prescription of drug therapy, is routinely performed only in special situations. As genetic testing becomes common in routine care settings, the traditional method of handling these test results will be inadequate. Current methods for performing medical and genetic tests typically utilize some form of personally identifiable information about each patient. The far-reaching implications of the information that genetic tests may provide to the patient and their family members coupled with the potential for abuse by insurers, employers and other entities may make any system that relies on personably identifiable inadequate. In addition, Federal legislation including, The Health Insurance Portability and Accountability Act (HIPAA) and various State laws specify stiff penalties for breech of such information. Additional, emphasis is also needed for systems and methods that assure that patients understand the terms of their informed consent for genetic testing and their rights to restrict future access to their genetic material and data by other parties.

Accordingly, a genetic testing system is needed that is particularly designed to test for genetic markers associated with drug safety and efficacy. The system also should provide the complete confidentiality that allows the user to be completely anonymous, while utilizing the advances and convenience of software and network technology. The collection of the specimen should be done in such a way that based upon the analysis of previously-collected genetic and phenotypic (medical history) data, samples of defined populations that meet specific criteria may be retested.

### SUMMARY OF THE INVENTION

Briefly, the invention includes systems, apparatus, and methods for anonymously testing and reporting drug efficacy or safety. One method described herein includes providing a DNA collection kit configured to enable collection and forwarding of a DNA test specimen for testing, where the kit includes a user results request with a user results identifier. The method further includes receiving the user results request and receiving test results for the DNA test specimen. The DNA test specimen may be identified by a computer-readable specimen identifier associated with the user results identifier. The method further includes matching the specimen identifier for the test results of the DNA test specimen with the associated user results request via the user results identifier and forwarding the test results in accordance with the user results request.

### BRIEF DESCRIPTION OF THE DRAWINGS

FIG. 1 is a schematic diagram of a genetic testing kit for anonymously submitting genetic test specimens for testing, including a test specimen, a user results request, and a provider results request according to one embodiment of the present invention.

FIG. 2 is an exemplary testing kit according to one embodiment of the present invention.

FIG. 3 illustrates one type of provider results request for the testing kit depicted in FIG. 2.

FIG. 4 illustrates one type of user results request for the testing kit depicted in FIG. 2.

FIG. 5 is a networked computer system for submitting genetic samples and receiving genetic testing results anonymously in accordance with one embodiment of the present invention.

FIG. 6 is a flowchart demonstrating the interaction between a user, a test manager, and a testing facility in accordance with one embodiment of the present invention.

FIG. 7 is a flowchart illustrating a method for anonymously testing and reporting drug efficacy or safety in accordance with one embodiment of the present invention.

FIG. 8 is a flowchart illustrating a method for creating and utilizing an anonymous genetic database in accordance with one embodiment of the present invention.

### DETAILED DESCRIPTION OF THE INVENTION

In view of the above, and in accordance with the present invention, a method, apparatus, and system for anonymously testing a genetic predisposition to medication safety and efficacy is provided. The present invention includes a method of privately submitting genetic samples for genetic and pharmacogenetic testing and allowing a user to anonymously and confidentially obtain the results of such tests.

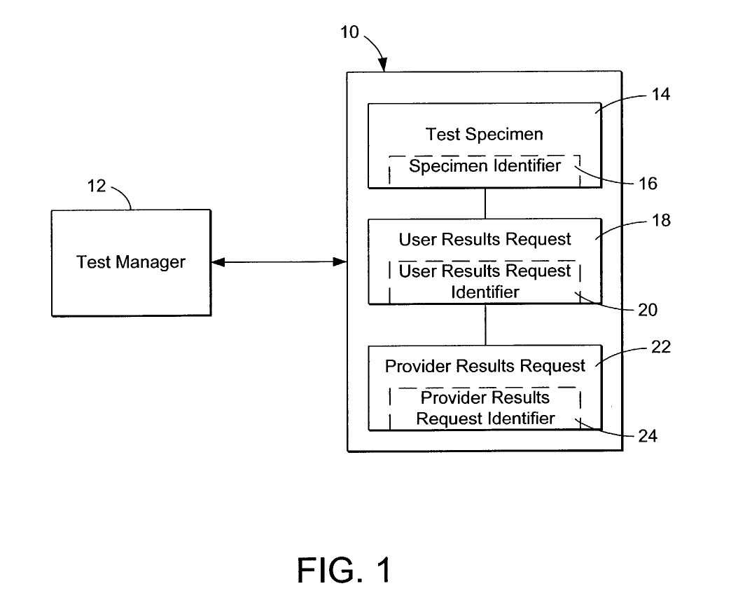

Referring initially to FIG. 1, a representative drug efficacy and safety testing kit is shown generally at 10. Genetic testing kit 10, also referred to herein as DNA collection kit and DNA testing kit, is configured to enable a user to collect and anonymously submit test specimens to a test manager 12 for testing. The kit may be used to test the safety and/or the effectiveness of a specific drug on an individual.

Kit 10 typically includes a method of collecting and forwarding a DNA test specimen 14 for testing. The DNA test specimen typically includes any body sample with DNA-containing cells. For example, the test specimen may be tissue or body fluid, including, but not limited to, blood, urine, buccal cells, semen, skin cells, hair, etc.

Test specimen 14 may be collected and stored for testing. A specimen identifier 16 identifies the test specimen. Specimen identifier 16 may be a computer-readable code unique to the test kit. The unique code may be any type of anonymous code, including, but not limited to, barcodes, computer generated digital codes, or any other type of randomly generated number code. Test specimen identifier 16 is used to track the test specimen and match the test specimen with the appropriate submitter/user. It should be appreciated that specimen identifier 16 may include modified specimen identifiers based on specimen identifier 16. As described in more detail below, these modified specimen identifiers may be derived from the original specimen identifier and operate to uniquely identify the specimen.

Kit 10 further includes a user results request device indicated generally at 18. The user results request includes a request for the test results to be sent to a user in a desired fashion, such as via an electronic file or via mail. For example, and as described in more detail below, user results request 18 may be contained within an executable software program or other device, such as a mail-in card. The user results request may include additional information regarding the kit or the testing. Moreover, the user results request may be linked or include releases and waivers, anonymous informed consent forms, survey questions, such as lifestyle questionnaires, etc.

User results request 18 includes a user results request identifier 20, also referred to herein, as user results identifier. The user results identifier 20 is specific to kit 10 and typically is linked or associated with specimen identifier 16, such that test manager 12 is able to match specimen identifier 16 with user results request identifier 20. User results identifier 20 may be a computer-readable code or other suitable code configured to be matched with specimen identifier 16.

Typically, a user submits user results request 18 to test manager 12 with the accompanying user results request identifier 20. In some embodiments, user results request identifier may be contained within the user results request and not easily accessible to the user. As described above, user results request identifier 20 is matchable with specimen identifier 16. By pairing the user results request identifier with the specimen identifier, test results for a test specimen may be forwarded to the appropriate user in accordance with the associated user results request. It should be appreciated that the test results may include one or more results. For example, the user results request may include a request for multiple tests results. In such an embodiment, the test results may include a full DNA screen and a list of drugs and their associated safety/effectiveness profiles for the tested DNA. Other test results may include only the results for a specific drug.

In some embodiments, a provider results request device may be included in kit 10. Similar to the user results request, provider results request 22 includes provider results request identifier 24. Provider results request identifier 24 corresponds with specimen identifier 16, and may correspond to a user results request identifier 20. When test manager 12 receives provider results request 22 and test specimen 14, test manager 12 is configured to match specimen identifier 16 with the provider results request identifier 24. Test manager 12 further may be configured to forward any test results associated with test specimen 14 to the matched provider, in accordance with provider results request 22.

Test manager 12 mediates the transactions between the user and/or provider and the testing facilities by linking specimen identifiers 16 with the user and provider results request identifiers 20, 24. Test manager 12 further may control and manage a genetic database containing test results and related information matched with user and provider result requests. It should be noted that the test manager may be a server, computing device, or program configured to manage the anonymous submittal and retrieval of genetic tests.

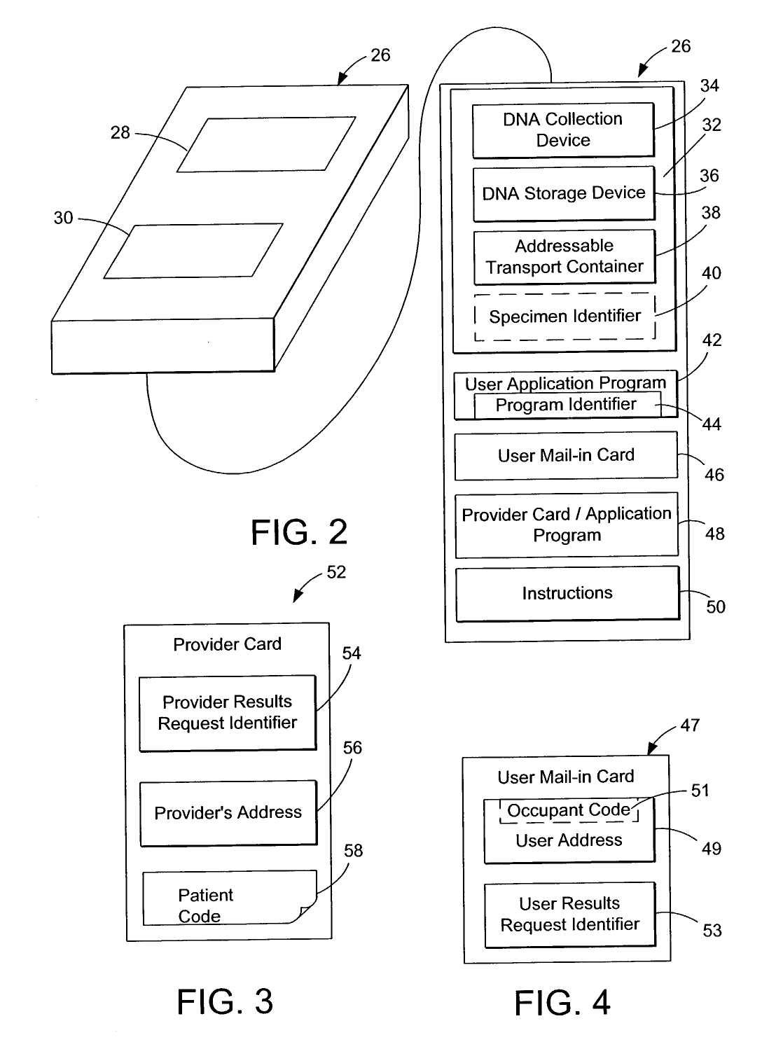

FIG. 2 illustrates an exemplary testing kit 26, in accordance with one embodiment of the present invention. The kit may be employed by a user to test their predisposition to safety and efficacy of a particular medication. The user may obtain the kit though a provider, a pharmacy, or other suitable distribution center.

The kit may be directed towards testing the safety and effectiveness of specific drugs or drug brands. By providing a drug-specific kit, the user and/or provider may be able to identify a specific drug's likely safety and effectiveness for the user based on their DNA. The kit enables a user to anonymously submit a DNA test specimen and anonymously retrieve his/her test results.

Depending on the type of drug, kit 26 may include specific drug and/or user identification information, as indicated at 28 and 30 in FIG. 2. The identification information may include the type of test kit, the specific drug to be tested for, specific information regarding the kit, a provider or patient, etc. The kit may also be uniquely identified via a kit code, which may enable the manufacturers to track use of the kit.

The test kit includes instrumentation to enable a patient to obtain a test specimen. For example, kit 26 typically includes a genetic specimen submission device, indicated generally at 32, configured to enable a user and/or a provider to collect and store a test specimen. In some embodiments, genetic specimen submission device 32 may include a DNA collection device, shown at 34, and a separate DNA storage device, shown at 36. In other embodiments, DNA collection device 34 may be integrated within the DNA storage device 36.

DNA collection device 34 includes the necessary equipment for a user/patient/provider to produce a suitable test specimen. DNA collection device 34 may be any suitable collection receptacle, including swabs, scrapers, test collection cards or vials, which may be used to collect tissue and/or body fluids. DNA collection device 34 also may include multiple collection aids that assist a user in producing the appropriate test sample. For example, when the body fluid to be analyzed or tested is blood, the kit may be equipped with an alcohol swab to clean an area of the skin, such as the tip of the middle or ring finger, before a blood sample is taken. The kit further may include a lancet (or plurality of lancets) that can puncture the skin so that blood may be acquired. The test kit further may include at least one bandage to protect the puncture after the blood sample is produced. In another embodiment, the kit may include a plurality of swabs configured to be used to collect squamous cells or other suitable specimens.

In some embodiments, the test specimen may be collected on a test collection card provided with the test kit. More particularly, the test collection card in one embodiment of the present invention is configured as a multi-part card. A first part of the card may include a specimen identifier, such as a barcode. The second part of the card may include special paper with specimen collection spots outlined thereon for the user to create specimens for testing. A prepared blood or test sample may be produced by placing enough blood on the specially designed blood specimen collection card to fill the specimen collection spots.

In another embodiment of the present invention, the sample card may include a plurality of separable segments. Each separable segment may include a specimen identifier, or identification code, and a test specimen. The identification code on each separable segment is identical. The separable segments may be routed to different facilities, including, but not limited to, designated laboratories, storage facilities, tracking facilities, etc.

DNA storage device 36 further may be placed into an addressable transport container 38. Alternatively, in some embodiments, DNA storage device 36 is configured to operate or serve as addressable transport container 38. Addressable transport container 38 typically includes the address of the required receiving facility. The receiving facility may be a management and routing facility (also referred to herein as a test manager), a research or testing facility, and/or a laboratory.

Regardless of the type of genetic specimen submission device 32, the device typically includes a specimen identifier 40 configured to identify the test specimen as connected with test kit 26. Thus, specimen identifier 40 may be included on an integrated DNA collection device and DNA storage device, on a separate DNA storage device or on addressable transport container 38.

Specimen identifier 40 may be pre-coded on genetic specimen submission device 32. For example, the kit may include pre-coded test specimen cards. Alternatively, a personal software program included with the kit may be configured to produce attachable specimen codes or specimen identifiers that may be affixed, or otherwise attached, to the genetic specimen submission device or addressable transport container by the user of the test kit. Typically, the specimen identifier is machine readable. For example, the specimen identifier may be a bar code or other suitable computer-readable code.

Specimen identifier 40 functions to identify the test specimen. As described briefly above, specimen identifier 40, as used herein, may include modified specimen identifiers. For example, the test manager may modify the original specimen identifier using a user's pin or other personal code to create a unique, undisclosed modified specimen identifier which is associated with both the user and the specimen and/or specimen test results. Such a modified specimen identifier may prevent an unauthorized user from copying the specimen identifier and accessing another user's results.

As described in more detail below, once the test specimen has been sent to a testing facility and analyzed, the test results are matched with specimen identifier 40 on genetic specimen submission device 32. The specimen identifier then can be further matched with results request identifiers (described in more detail below), such that the associated test results can be sent to the appropriate party/parties.

Kit 26 further may include a user results request device. In some embodiments, such as the embodiment shown in FIG. 2, the user results request device may be a user-application program indicated at 42. A user may load the user-application program, or executable software program, on a personal computer and/or run the program from a mass storage device. For example, the software program may be contained on a mass storage device, such as a CD/ROM. Alternatively, the software may be stored on a remote computer and may be accessible through a computer network, such as the Internet.

To access test results, the user may create or activate a personal electronic results file via the software program. The program may link with a result database such that a user may download his/her results to a personal computer of their choice. The link may be through any suitable network, including, but not limited to, public networks, such as the Internet, and/or private networks. If a public network is used, security features may be provided to ensure the confidentiality of the test results. Hence, an electronic file may be created and subsequently accessed anonymously and remotely from any suitable computing device, such as a personal computer, via the software program. In some embodiments, a user may define a pin or key code to limit access to the personal electronic results file. Typically, such a pin or key code is a user-selectable code.

User-application program 42 contains a user results request identifier, such as a program identifier 44, which enables the confidential match of the user's test results with user-application program 42. Program identifier 44 is unique to kit 26 and may be pre-associated with specimen identifier 40. Program identifier 44 may be a computer-readable code included within user-application program 42. For example, the software may enable a user to create a personal electronic file, which may include a computer-readable user results request identifier or code. The computer-readable user results request identifier may be manufactured within the software program itself, associated with the software program, randomly generated within the software program or created by the user. The personal electronic file maintains the anonymity/confidentiality of the person taking and seeking the test results.

In other embodiments, the user results request device may be a user mail-in card, indicated at 46. User mail-in card may be provided for users who do not have access to a computer or who would prefer not to use the computer. Similar to user-application program 42, user mail-in card 46 includes a user results request identifier pre-associated with specimen identifier 40 and used to pair test results for the specimen with the user.

A provider results request device may also be included within kit 26. For example, a provider card (described below) and/or a provider application program, indicated at 48, may be included or associated with kit 26. When the provider results request is contained within a provider application program, the provider may create a provider electronic file that is connected via the network to the test manager. The provider application program may be contained on a mass storage device or run from the provider computer. The provider application program may be a web-based or Internet-based application. The test manager may then send and receive messages via the provider electronic file. As described above, the provider results request device typically includes a provider results request identifier associated with the specimen identifier. The provider results request identifier may be matched to the specimen identifier such that the appropriate test results for a specific user may be forwarded to the provider. Thus, test results with a specimen identifier may be matched with a provider results request identifier and then forwarded, automatically, or upon request, to the provider electronic file.

Kit 26 may include additional components, including, but not limited to, instructions, such as instruction cards, indicated at 50, information regarding the testing kit and/or testing process, information regarding the drug, consent forms, questionnaires, etc. Instructions 50 may include, but are not limited to, directions informing the user and/or provider how to collect a DNA sample using the DNA collection device, how to store the DNA sample in the DNA storage device, how to send the DNA sample for testing, how to execute the application program, and/or how to retrieve test results using the application program.

FIG. 3 illustrates, in more detail, one type of provider results request device. Specifically, the provider results request device in FIG. 3 is a provider card 52. Provider card 52 is illustrated as a mail-in card, which includes a request to send test results for the user to the provider address on the card. Provider card 52 includes a provider results request identifier, indicated at 54, a provider address 56, and a patient code-58. Provider results request identifier 54 is pre-associated with the specimen identifier for the test specimen in order to enable matching of the test results with the provider. The provider may fill in or attach a label to provider card 52 with the provider's address. In some embodiments, the provider's address may be preprinted on provider card 52 for the specific provider.

Patient code 58 may be a human-readable code. The code enables the provider to match the test results received from the test manager with a patient. In some embodiments, patient code 58 may be removable such that a provider may attach the patient code to the appropriate file. For example, the patient code may be a peel-off or tear-off portion that the provider may attach to a patient file.

FIG. 4 illustrates a user mail-in card 47 which may operate as a user results request. User mail-in card 47 typically includes a field into which a user may supply their address 49 and an occupant code 51. The user does not need to include their name, or personal identifying information, other than a user-selected address. Excluding the user's name enables the mail-in card to maintain the user's anonymity. Instead of the user's name, the user may select an occupant code 51 to identify the user. User mail-in card 47 is matched with the specimen via a user results request identifier 53. User results request identifier 53 may be a computer readable code that is preprinted on user mail-in card 47.

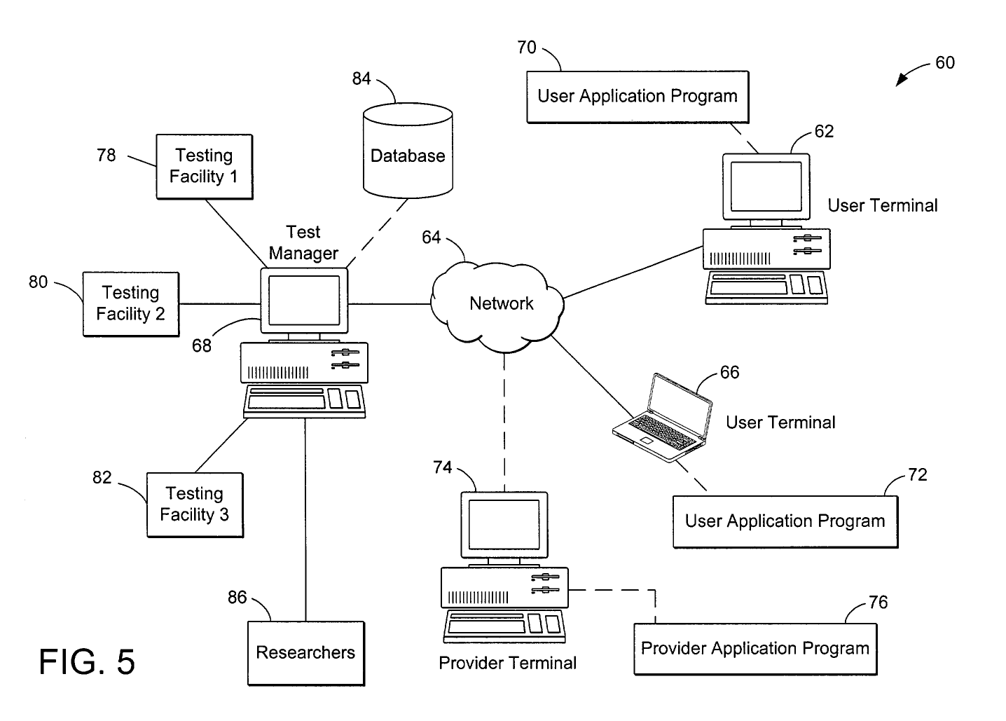

FIG. 5 illustrates a networked computer system for submitting genetic samples and receiving genetic testing results anonymously according to one embodiment of the present invention. Such a networked computer system is indicated generally at 60. Networked computer system 60 enables a user and/or a provider to collect, submit, and receive genetic test results anonymously.

As illustrated, networked computer system 60 typically includes at least one user terminal 62 linked to network 64. User terminal 62 is shown as a personal desktop computer, however, it should be appreciated that computer 62 may be any suitable computing device that is capable of linking to the network and receiving data. For example, computer 62 may be a hand-held computer, a laptop computer (as illustrated at 66), a portable computer, a server, or a series of linked computers.

It should be noted that multiple users may link to network 64. For example, in the illustrated system, a first user terminal is shown at 62 and a second user terminal is shown at 66. Each user terminal 62, 66 is configured to execute a user-application program, indicated respectively at 70, 72, which enables the user to link with network 64 and test manager 68. The user-application program may be stored on a mass storage device, or may be run from a user terminal. In some embodiments, the user-application program may be a web-based, or Internet-based application. Network 64 may be any suitable type of communications link, such as a local area network (LAN) or a wide area network (WAN). The WAN may include a public network, such as the Internet.

Provider terminals 74 may also be linked to network 64. Such provider terminals may be configured to execute provider application programs 76, which enable a provider to link with network 64 and test manager 68. As described above, provider application program 76 contains a provider results identifier that is configured to be matched with the specimen identifier to enable results for a specific test specimen to be forwarded to the provider.

User terminal 62, 66 and provider terminal 74 are linked via network 64 to test manager 68. Test manager 68 is configured to receive user results request with user results request identifiers and provider results request with provider results request identifiers. Test manager 68 matches the results request identifiers with the specimen identifier.

In some embodiments, test manager 68 is configured to receive the test specimens from the user/provider and route the test specimens to the appropriate testing facilities. Test manager 68 may record the specimen identifiers within a results retrieval system, or other suitable database, indicated at 84, and then route the test specimen to the appropriate laboratory. Test results received from a testing facility may include codes that may be associated with the specimen identifiers such that the test results may be matched to the specimen identifier and entered into the results retrieval system. For example, test manager 68 may receive a test specimen with a specimen identifier. Test manager 68 may recode the test specimen prior to sending the test specimen to a testing facility. The testing facility may send test results with the associated test manager assigned code back to the test manager. The test manager may then match the test manager assigned code with the original specimen identifier or modified specimen identifier.

Multiple testing facilities 78, 80, and 82 may be linked to test manager 68. Testing facilities 78, 80 and 82 may be linked to test manager 68 via a private or public network (not shown), such as the Internet. The testing facilities may be research facilities, laboratories, or other facilities capable of conducting genetic tests. The testing facilities may include on-site scientists or researchers, groups of scientists or researchers, or other suitable facilities. It should be appreciated that in some embodiments, a testing facility or facilities may operate as test manager 68.

Upon receipt of the test specimen, the testing facility analyzes the test specimen as requested. For example, the testing facility may analyze the test specimen to determine if specific genetic markers are present related to the patient's predisposition to a particular medication being safe and/or effective for them. The test results also may be referred to herein as genotype data. Once the analyzation step is completed, the testing facility may input the test results into a computer database that is linked to test manager 68.

Test manager 68 manages forwarding of the test results received from testing facilities 78, 80, and 82 to the appropriate party/parties. Typically, test manager 68 matches the specimen identifiers for each test specimen with a user results request identifier and/or a provider results request identifier. For example, the user may have loaded user-application program 70 onto user terminal 62 creating a personal electronic file identified by a user results request identifier. Test manager 68 may then forward the test results, or a message that the test results are available, to the personal electronic file having the matching user results request identifier. The user may then anonymously access the test results by accessing his/her personal electronic file. The provider may similarly access the test results.

Test manager 68 may retain a copy of the test results in database 84. Researchers, testing facilities, etc., as indicated at 86, may be linked to test manager 68 and use database 84 for research and other studies. Test manager 68 may limit access to portions of database 84, ensuring the privacy of the users who submitted genetic specimens.

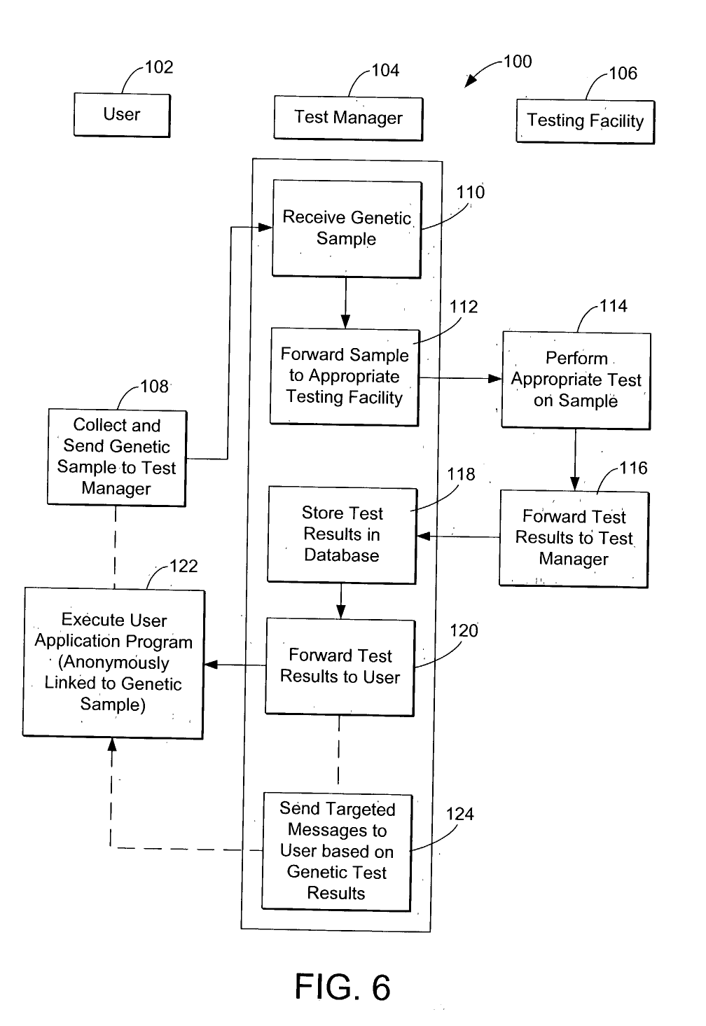

FIG. 6 illustrates at 100 the relationship between a user 102, a test manager 104 and a testing facility 106. Specifically, as shown at 108, the user collects and sends a genetic test sample or specimen. In some embodiments, a provider may send a patient's genetic test sample to test manager 104.

In some embodiments, test manager 104 receives the genetic sample, as illustrated at 110, and forwards the sample to the appropriate testing facility 106, at 112. Testing facility 106 receives the sample and performs the appropriate test on the sample, at 114 and then forwards the test results to test manager 104.

Test manager 104 may receive the test results, or genotype data, and store such data in a genetic database, at 118. Test manager 104 also may forward the test results, or genotype data, to the user, at 120, in accordance with the user results request. As described above, the user results request may be contained within an application program that may be executed by user 102. Execution of the user-application program, at 122, enables test manager 104 to forward the test results via a network to user 102. Execution of the user-application program may include loading or downloading an associated program and/or running a program from a mass storage device, such as a storage disk.

Typically, execution of the user-application program includes creating a personal electronic file accessible via a network. In such embodiments, a user may electronically access his/her test results by accessing his/her personal electronic file. As described above, the personal electronic file may include a code, such as the user results request identifier that is matched to the specimen code or specimen identifier, to enable the test manager to forward the corresponding test results to the appropriate personal electronic file. Alternatively, test manager 104 may mail the test results to the appropriate user 102.

In some embodiments, test manager 104 may send additional messages, also referred to herein, as research inquires, to user 102, at 124. The messages may be based on the user's genetic test results and/or other user-specific information, such as questionnaire data or clinical trial information. For example, targeted messages based on genotypic and phenotypic data may be sent by the test manager, laboratory, or other party to the user's software or personal electronic file. The messages, or research inquires, may include, but are not limited to, informed consent requests, tests regarding whether the user understands the informed consent requests, consent requests for additional genetic testing, clinical trial enrollment forms, medication adherence and compliance surveys, medication side-effect surveys, adverse drug reaction surveys, post-marketing (phase IV) surveillance forms, etc. The user may access the messages in a manner similar to the method used to access his/her test results. Additionally, the user's software may be configured to permit the user to reply to any such messages sent by the test manager, laboratory or other party. Such messages may include responses to the requests for additional test and/or information.

For example, in some embodiments, the user results request may include an informed consent agreement. The test manager may send messages to the user regarding their understanding of the informed consent agreement. The user may be able to alter the contents of the informed consent agreement to conform to their understanding and/or desire. For example, the user may be able to retain control, by selecting or approving any proposed research or testing, commercial or otherwise, of his/her genetic sample and genetic data.

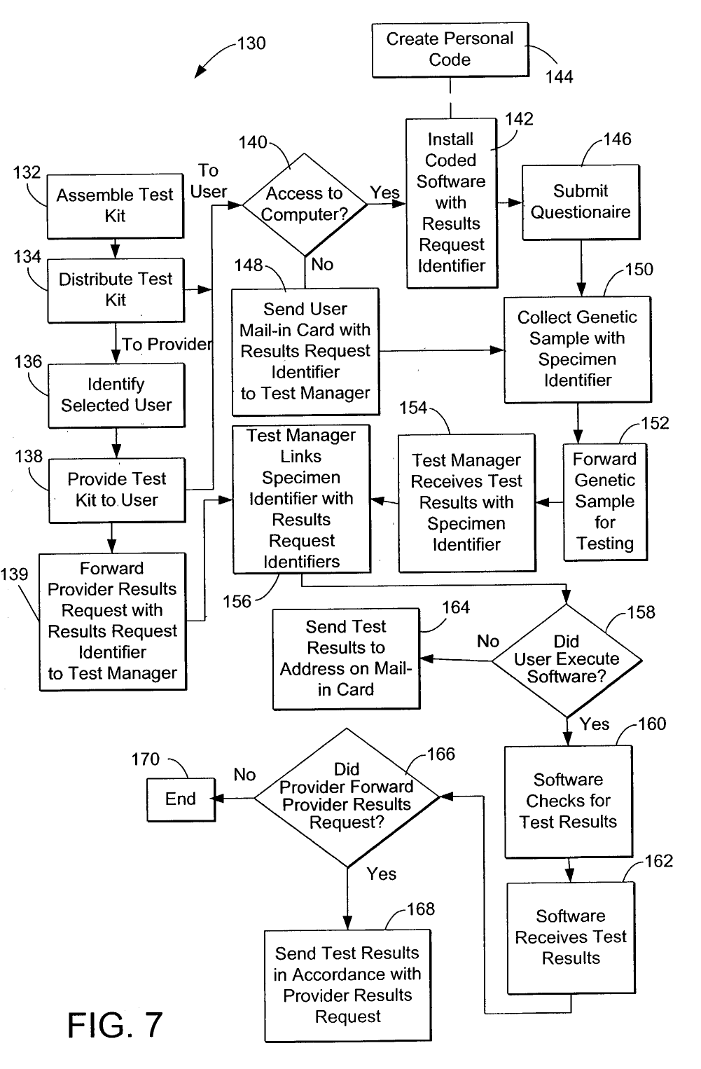

FIG. 7 further illustrates a method at 130 for anonymously testing and reporting drug efficacy or safety, in accordance with one embodiment of the present invention. The method includes assembling test kits, at 132 and distributing the test kits, at 134. The test kits may be distributed to providers and/or to retail establishments, such as pharmacies, drug stores, grocery stores, etc.

In some embodiments, assembling the kits includes generating a matched specimen identifier and result request identifiers for each kit. The matched identifiers may be maintained in a database managed by the test manager. In other embodiments, the specimen identifiers and result request identifiers are generated after executing the user software program. Such identifiers may be sent to the test manager.

Providers may, prior to prescribing a drug to a user, wish to understand the effectiveness and safety of the drug on the particular user. Thus, a provider may identify a user, at 136, and distribute the kit to the user, at 138. In some embodiments, the kit may include a provider results request with a provider results request identifier. The provider may forward the provider results request with the provider results request identifier to the test manager, at 139.

As described above, the genetic testing kit may include user software, which enables a user to anonymously request the test results. A user who has access to a computer, at 140, may install the software containing a coded user results request identifier, at 142. Although illustrated prior to the user collecting the test specimen, the user may install or execute the software before or after collecting and sending the sample to the test manager or testing facility.

The user may be able to create a security code or personal pin/key to limit access to the software, at 144. The security codes may be any suitable personal code that may enable a user to ensure that the software has not been previously loaded. The security codes further may enable the user to access a personal electronic file created using the software from a different computing device. Additionally, the security codes may allow multiple test results to be compiled together.

In some embodiments, a questionnaire may be provided, as shown at 146. For example, the software may include a personal questionnaire, including lifestyle questions. The answers to such questionnaires may be maintained in an anonymous database. User information or data, such as responses to questionnaires, may be transferred to the test manager by the personal software.

If a user opts to not execute the software, the user may send in a user mail-in card with the results request identifier to the test manager, at 148. Questionnaires may be provided, which are intended to be mailed to the test manager or other suitable facility with the mail-in card.

The method further includes, a user collecting a genetic sample, at 150 and forwarding the genetic sample for testing, at 152. The genetic sample may be forwarded to a test manager who routes the genetic sample to the appropriate testing facility. Alternatively, the user may send the genetic sample directly to a testing facility. The testing facility may route the specimen to a more specific laboratory depending on the type of test specimen. The testing facility performs the appropriate analysis and forwards the results to the test manager. In some embodiments, the testing facility enters the test results into a networked computer system. The test results may be identified by the specimen identifier that originally accompanied the test specimen, at 154.

Upon receipt of the test results, the test manager links the specimen identifier with the coded results request, at 156. As used herein, a coded results request is a combination of the results request (such as a user results request and/or a provider results request) and the associated results request identifiers. Thus, the specimen identifier may be matched with the user results request identifier and/or the provider results request identifier. The test results are then forwarded to the user and/or provider in accordance with the results requests.

For example, if the user executed the user software, at 158, the test results may be accessible via the software. In some embodiments, the user may use the software to check if the test results are available. The user may then access the test results through the computer, at 162. In other embodiments, the software may automatically check whether the test results are available, at 160. For example, the software may perform an automated periodic request whether the test results are available. When available, the test results may be automatically sent and received by the user's local computer operating the software. In some embodiments, once the test results are received, the results reporting portion of the software may be disabled and the message system between the software and the test manager may be enabled. Alternatively, if the user did not execute the software, the user may receive the test results at the address on the mail-in card, at 164.

Similarly, if the provider forwarded a provider results request, at 166, the test results may be sent to the provider in accordance with the provider results request, at 168. For example, if the provider executed a provider application program, the test results may be electronically sent to the provider via a computer network. In some embodiments, the tests results may be sent automatically, while in other embodiments, the user may have to request the test results. Alternatively, if the provider mailed in a provider card, the results may be mailed to the provider. The provider may be able to match the test results with a patient specific code.

Accordingly, as set forth above, multiple methods are provided. For example, a method for anonymously testing and reporting drug efficacy or safety is provided. The method includes providing a DNA collection kit configured to enable collection and forwarding of a DNA test specimen for testing, wherein the kit includes a user results request with a user results identifier. The method further includes receiving the user results request, receiving test results for the DNA test specimen, wherein the DNA test specimen is identified by a computer-readable specimen identifier associated with the user results identifier, and matching the specimen identifier for the test results of the DNA test specimen with the associated user results request via the user results identifier. The test results may be forwarded in accordance with the user results request.

Another method for anonymously testing and reporting drug efficacy or safety includes providing a DNA collection kit configured to enable collection and forwarding of a DNA test specimen marked with a specimen identifier. The kit includes a user-application program with a program identifier, where the program identifier corresponds with the specimen identifier. The method further includes receiving the DNA specimen, routing the DNA specimen to a selected testing facility, receiving test results for the DNA specimen from the testing facility, identifying the user-application program associated with the test results by matching the specimen identifier with the program identifier, and forwarding test results for the DNA specimen to the identified user-application program.

Another method, described herein, includes receiving genetic test results from a testing facility identified by a unique computer-readable specimen code from a user's test specimen, storing the genetic test results in a DNA database, and receiving a coded results request that corresponds to the specimen code to receive the genetic test results. The method further includes matching the coded results request with the genetic test results via the computer readable specimen code and providing the genetic test results in accordance with the matched coded results request.

A method of anonymously submitting and receiving genetic test results is further provided where the method includes providing a DNA collection kit configured to enable collection and forwarding of a DNA test specimen to a testing facility. The DNA test specimen may be identified via a specimen identifier. The method further includes receiving genotype data from the testing facility for the DNA test specimen, and matching the genotype data with phenotype data, such as the user results request and user results request identifier. Matching of the genotype data with the phenotype data may include matching the specimen identifier with a results request identifier included within the phenotype data.

Another method includes receiving a plurality of genetic test results, where each test result is identified by a specimen identifier that corresponds to a coded result request. The method also includes receiving a plurality of coded result requests, matching each specimen identifier with the corresponding coded result request, storing the genetic test results in a database with the specimen identifier and the matched coded result request, and communicating a specific genetic test result in accordance with the matched coded result request.

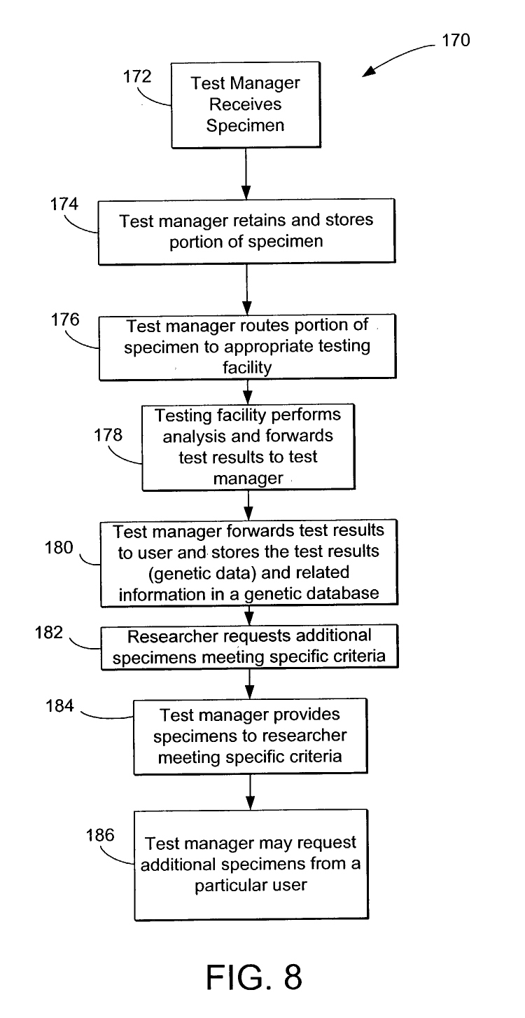

FIG. 8 further illustrates a method 170 according to another embodiment of the present invention. Method 170 includes steps for creating and utilizing an anonymous genetic database. Specifically, the method enables representatives from organizations, such as biotechnology and pharmaceutical companies, to anonymously communicate with people who meet specific genotypic or phenotypic criteria. Such communication may enable supplementary testing of particular test specimens for research purposes. Additionally, the method enables a laboratory or research facility to re-test samples for defined populations based upon analyzed genetic and phenotypic data.

The method includes a test manager receiving a genetic test specimen from a user and/or provider, at 172. The test manager may be a routing and management facility and/or a laboratory or research facility. The test manager may retain and store a first portion of the genetic test specimen, at 174 and route a second portion of the genetic test specimen to the appropriate testing facility, at 176. The testing facility performs its analysis and forwards the test results to the test manager, at 178.

The test manager forwards the results to the user and also stores the test results and related information in a genetic database, at 180. The related information may include answers to a questionnaire provided to the user and/or clinical trial information. Researchers, laboratories, or testing facilities may request additional specimens meeting specific criteria from the test manager, at 182. The test manager may search the database to identify specimens with the desired criteria and provide such specimens to the researchers, at 184. In some embodiments, the test manager may request information or additional specimens from a user to aid the researcher, at 186. For example, the test manager may request additional test specimens and/or data from a user that meets selected criteria requested by a laboratory, researcher, or testing facility. Such data may include, but is not limited to, genotypic, phenotypic, and family history data. The test manager further may request whether a user wants to participate in a clinical trial. The user may respond to such requests via a personal electronic file established upon request of the test results.

While various alternative embodiments and arrangements for anonymously testing and reporting drug efficacy and safety have been shown and described above, it will be appreciated by those skilled in the art that numerous other embodiments, arrangements, and modifications are possible and are within the scope of the invention. Thus, although the present invention has been disclosed in specific embodiments thereof, the specific embodiments are not to be considered in a limiting sense, because numerous variations are possible. The subject matter of the invention includes all novel and nonobvious combinations and subcombinations of the various elements, features, functions, and/or properties disclosed herein.

The following claims particularly point out certain combinations and subcombinations regarded as novel and nonobvious. These claims may refer to “an” element or “a first” element or the equivalent thereof. Such claims should be understood to include incorporation of one or more such elements, neither requiring, nor excluding two or more such elements. Other combinations and subcombinations of features, functions, elements, and/or properties may be claimed through amendment of the present claims or through presentation of new claims in this or a related application. Such claims, whether broader, narrower, equal, or different in scope to the original claims, also are regarded as included within the subject matter of the invention of the present disclosure.

We claim:

1. A method for anonymously testing and reporting drug efficacy or safety, comprising:
providing a DNA collection kit configured to enable collection and forwarding of a DNA test specimen for drug efficacy or safety testing, wherein the kit includes a user results request with a user results identifier;
receiving the user results request;
receiving test results for the DNA test specimen, wherein the DNA test specimen is identified by a computer-readable specimen identifier associated with the user results identifier;
matching the specimen identifier for the test results of the DNA test specimen with the associated user results request via the user results identifier; and
forwarding the test results in accordance with the user results request.

2. The method of claim 1, further comprising prior to receiving the test results for the DNA test specimen, receiving the DNA test specimen from the user and routing the DNA test specimen to a selected testing facility.

3. The method of claim 1, wherein the user results request is contained within a user-application program and the user results identifier is a user program identifier.

4. The method of claim 3, wherein forwarding test results in accordance with the user results request includes automatically forwarding the test results to the user-application program via a network.

5. The method of claim 3, wherein the user-application program is a web-based application.

6. The method of claim 1, wherein the user results request is a mail-in card including a user address, and where forwarding test results in accordance with the user results request includes sending test results to the user address.

7. The method of claim 1, wherein the DNA collection kit includes a DNA collection device marked with the specimen identifier.

8. The method of claim 1, wherein the DNA collection kit includes a DNA storage device marked with the specimen identifier.

9. The method of claim 1, wherein the DNA collection kit includes a DNA transport device marked with the specimen identifier.

10. The method of claim 1, wherein the DNA collection kit includes a provider results request with a provider results request identifier that corresponds with the specimen identifier.

11. The method of claim 10, further comprising matching the specimen identifier for the test results of the DNA test specimen with the provider results request identifier; and
forwarding test results in accordance with the provider results request.

12. The method of claim 11, wherein the provider results request is contained within a provider application program, and where forwarding test results in accordance with the provider results request includes automatically forwarding test results to the provider application program.

13. The method of claim 11, wherein the provider results request is a mail-in card including a provider address, and where forwarding test results in accordance with the provider results request includes sending test results to the provider address.

14. The method of claim 1, further comprising sending targeted messages to a user based on the test results in accordance with the user results request.

15. A method for anonymously testing and reporting drug efficacy or safety, comprising:
providing a DNA collection kit configured to enable collection and forwarding of a DNA test specimen marked with a specimen identifier; wherein the kit includes a user-application program with a program identifier, where the program identifier corresponds with the specimen identifier;
receiving the DNA specimen;
routing the DNA specimen to a selected testing facility;
receiving test results for the DNA specimen from the testing facility;
identifying the user-application program associated with the test results by matching the specimen identifier with the program identifier; and
forwarding test results for the DNA specimen to the identified user-application program.

16. The method of claim 15, further comprising prior to forwarding the test results for the DNA specimen to the identified user-application program, receiving a query from the user-application program requesting test results.

17. The method of claim 15, wherein the specimen identifier is a bar code.

18. The method of claim 15, wherein the DNA collection kit further includes a provider results request with a provider results request identifier that corresponds with the specimen identifier, and the method further includes receiving the provider results request, matching the provider identifier with the specimen identifier, and forwarding the test results in accordance with the provider results request.

19. The method of claim 18, wherein the provider results request includes a provider application program, and where forwarding the test results includes sending the test results to the matched provider application program via a network.

20. The method of claim 15, further comprising storing the specimen identifier, the program identifier, and the test results for the DNA specimen in a database.

21. The method of claim 20, further comprising providing research access to at least a portion of the database, and subsequently sending a research inquiry to a selected user-application program regarding the DNA specimen.

22. The method of claim 15, wherein the test results include information related to the efficacy of a specific drug for a user originating the DNA specimen.

23. The method of claim 15, wherein the test results include information related to the effects of a specific drug for a user originating the DNA specimen.

24. A method of providing anonymous genetic test results, the method comprising:
receiving genetic test results from a testing facility identified by a unique computer-readable specimen code from a user's test specimen;
storing the genetic test results in a DNA database;
receiving a coded results request that corresponds to the specimen code to receive the genetic test results;
matching the coded results request with the genetic test results via the computer readable specimen code; and
providing the genetic test results in accordance with the matched coded results request.

25. The method of claim 24, wherein the coded results request includes a request to receive the genetic test results electronically, and where providing the genetic test results in accordance with the matched coded results request includes automatically electronically forwarding the genetic test results in accordance with the coded results request.

26. The method of claim 24, wherein the coded results request includes a user results request and a user results request identifier.

27. The method of claim 24, wherein the coded results request includes a provider results request and a provider results request identifier.

28. A method of creating an anonymous DNA database, the method comprising:
receiving a plurality of genetic test results, each test result identified by a specimen identifier that corresponds to a coded result request;
receiving a plurality of coded result requests;
matching each specimen identifier with the corresponding coded result request;
storing the genetic test results in a database with the specimen identifier and the matched coded result request; and
communicating a specific genetic test result in accordance with the matched coded result request.

29. The method of claim 28, wherein communicating a specific genetic test result in accordance with the matched coded result request includes providing the test results to at least one of a user and a provider.

30. The method of claim 28, wherein communicating a specific genetic test result in accordance with the matched coded result request includes mailing the test results.

31. The method of claim 28, wherein communicating a specific genetic test result in accordance with the matched coded result includes electronically providing the test results via a network.

32. The method of claim 28, wherein prior to receiving a plurality of genetic test results, the method includes:
receiving a plurality of specimens, each specimen identified by the specimen identifier,
cataloging each of the specimens according to the specimen identifier in the database;
forwarding each of the specimens for testing.

33. The method of claim 32, further comprising storing a portion of each of the specimens prior to forwarding each of the specimens for testing.

34. The method of claim 28, further comprising receiving responses to a questionnaire associated with a coded result request and storing the responses in the database with the associated coded result request.

35. A method of anonymously submitting and receiving genetic test results, the method comprising:
providing a DNA collection kit configured to enable collection and forwarding of a DNA test specimen to a testing facility, where the DNA test specimen is identified via a specimen identifier;
receiving genotype data from the testing facility for the DNA test specimen; and
matching the genotype data with phenotype data by matching the specimen identifier with a results request identifier included within the phenotype data.

36. A kit for testing drug efficacy or safety, comprising:
a DNA collection device adapted to enable a user to collect a DNA sample;
a DNA storage device configured to store the DNA sample, wherein at least one of the DNA collection device and the DNA storage device has a computer readable specimen identifier;
an addressable transport container configured to send the DNA sample for testing; and
an application program configured to be executed on a networked computer, wherein the application program includes a program identifier matched with the computer readable specimen identifier to facilitate test results retrieval.

37. The kit of claim 36, wherein the DNA storage device is configured to serve as the addressable transport container.

38. The kit of claim 36, further including instructions that direct the user how to collect a DNA sample using the DNA collection device, how to store the DNA sample in the DNA storage device, how to send the DNA sample for testing, and how to retrieve test results using the application program.

39. The kit of claim 36, further including a provider test results request having a provider results request identifier, where the provider results request identifier corresponds to the computer readable specimen identifier such that test results associated with the specimen identifier may be matched with the provider results request identifier and forwarded to a provider in accordance with the provider results request.

40. A program storage device readable by a machine, the storage device tangibly embodying a program of instructions executable by the machine to perform a method for anonymously testing drug efficacy or safety, the method comprising;
receiving a genetic test result from a testing facility, where the genetic test result is identified by a computer-readable specimen code;
storing the genetic test result in a database;
receiving a computer-coded request to receive the genetic test result, where the computer-coded request is associated with the specimen code;
matching the computer-coded request with the specimen code; and
providing the genetic test result electronically upon matching the computer-coded request with the specimen code.

41. A program storage device readable by a machine, the storage device tangibly embodying a program of instructions executable by the machine to perform a method for communicating with a test manager, the method comprising:
establishing an electronic link to a genetic test manager;
electronically submitting a results request having a results request identifier to the test manager, wherein the results request identifier is associated with test results for a test specimen;
polling the test manager for test results for the test specimen; and
receiving test results for the test specimen when the test results for the associated test specimen are submitted to the test manager.

42. A system for anonymously conducting genetic testing, the system comprising:
a network;
a processor linked to the network adapted to execute a user-application program having a program identifier, where the program identifier is linked with a DNA test specimen; and
a test manager linked to the network configured to match the DNA test specimen with the program identifier to enable communication between the user-application program and the test manager regarding the DNA test specimen.

43. The system of claim 42, wherein the test manager is further configured to receive and route the DNA test specimen to a testing facility.

44. The system of claim 42, wherein the test manager is further configured to receive test results for the DNA test specimen from a testing facility and automatically forward the test results to the user-application program.

45. The system of claim 42, wherein the program identifier is encoded within the user-application program.

46. A system for anonymously testing drug efficacy or safety, the system comprising:
a DNA collection kit adapted to enable a user to collect a DNA specimen having a predetermined specimen identifier, wherein the kit includes a user results request with a user results request identifier, and a provider results request with a provider results request identifier; and
a test manager configured to link the specimen identifier with the user results request identifier and the provider results request identifier, the test manager further configured to send test results to both the user, in accordance with the user results request, and the provider, in accordance with the provider results request.
